(19) United States
(12) Patent Application Publication
(10) Pub. No.: US 2011/0178012 A1
(43) Pub. Date: Jul. 21, 2011

(54) USE OF HUMAN CHORIONIC GONADOTROPIN ORALLY FOR THE TREATMENT OF OVERWEIGHT (OBESITY) ASSOCIATED WITH HIGH BLOOD TENSION, NON-INSULIN-DEPENDANT DIABETES, HYPERCHOLESTEROLEMIA, DYSLIPIDEMIAS AND LIPODYSTROPHY.

(76) Inventor: DANIEL OSCAR BELLUSCIO, Capital Federal (AR)

(21) Appl. No.: 13/078,474

(22) Filed: Apr. 1, 2011

Related U.S. Application Data
(63) Continuation of application No. 11/826,214, filed on Jul. 13, 2007, now abandoned.

(30) Foreign Application Priority Data
Jul. 21, 2006 (AR) P20060103147

(51) Int. Cl. A61K 38/09; A61P 3/00; A61P 9/12

(52) U.S. Cl. 514/10.3

(57) ABSTRACT

Human chorionic gonadotropin administered by intramuscular route is traditionally used for the treatment of sterility and cryptorchidism. After numerous tests performed, a compound to be administered orally—sublingual—has been developed with such drug to treat patients suffering from overweight associated with high blood tension, non-insulin-dependant diabetes, hypercholesterolemia, dyslipidemias and lipodystrophy. It was determined that hCG acts satisfactorily upon high blood tension, non-insulin-dependant diabetes, hypercholesterolemia, dyslipidemias and lipodystrophy.

### DETAILED DESCRIPTION OF INVENTION

The present application is filed as a continuation application and claims priority of application Ser. No. 11/826,214.

This invention relates to “THE USE OF HUMAN CHORIONIC GONADOTROPIN ORALLY FOR THE TREATMENT OF OVERWEIGHT (OBESITY) ASSOCIATED WITH HIGH BLOOD TENSION, NON-INSULIN-DEPENDANT DIABETES, HYPERCHOLESTEROLEMIA, DYSLIPIDEMIAS AND LIPODYSTROPHY.”

### SCOPE

The compound is to be used for controlled treatment of overweight associated with high blood tension, non-insulin-dependant diabetes, hypercholesterolemia, dyslipidemias and lipodystrophy.

### CURRENT TECHNIQUE

In the current technique, Human Chorionic Gonadotropin is administered by intramuscular route to treat sterility and cryptorchidism disorders.

### PROBLEM TO BE SOLVED

When obese persons are healthy, overweight treatment is easier to administer through diets to be followed without difficulties by patients. However, where overweight is associated with health problems like high blood tension, diabetes, hypercholesterolemia, dyslipidemias and lipodystrophy, treatments are difficult and administration of additional drugs as coadjuvant therapy is necessary.

### THIS INVENTION CAN SOLVE THE PROBLEM

After several assays testing, selecting and discarding drugs, Human Chorionic Gonadotropin was determined to act satisfactorily to solve the problem. On one hand, this drug acts inhibiting fat cell (adipocytes) lipogenesis, and on the other, Beta endorphin in gonadotropin molecule has action on the hypothalamus, thus allowing an improvement in patient's clinical condition during treatment, with rapid improvement on glycemia, lipid, cholesterol and blood tension levels. Finally, the most suitable administration form, and the one which showed the best results as well, was determined to be sublingual form.

### ADVANTAGES

An advantage of this invention is the successful treatment of obesity in patients with high blood tension, non-insulin-dependant diabetes, hypercholesterolemia, dyslipidemias and lipodystrophy. The advantages of this invention, which should not be limited to the description above, will become more apparent and the invention itself better understood by reference to the following example of a preferred embodiment.

### DESCRIPTION OF EMBODIMENT

“The use of human chorionic gonadotropin orally for the treatment of overweight (obesity) associated with high blood tension, non-insulin-dependant diabetes, hypercholesterolemia, dyslipidemias and lipodystrophy” is represented by the following formulation: Powder Human Chorionic Gonadotropin dissolved in physiological saline and administered placing it under the tongue. A preferred drug formulation consists of 500 international units of powder Human Chorionic Gonadotropin per milliliter solution.

### ACTION

As stated elsewhere, the drug action inhibits fat cell (adipocytes) lipogenesis, and on the other hand, Beta endorphin in gonadotropin molecule has action on the hypothalamus, thus allowing an improvement in patient's clinical condition during treatment, with rapid improvement on glycemia, lipid, cholesterol and blood tension levels.

The advantages of this invention are plain from the description above, representing a beneficial technological improvement that warrants the inclusion of the invention in the law with the pertinent legal protection as per the appended claims.

1. A method of treating a human patient suffering from high blood tension, non insulin dependant diabetes, hypercholesterolemia or lipodystrophy comprising the oral administration of an effective amount of a solution of human chorionic gonadotropin (hCG), to said human patient in need thereof.

2. The method of claim 1 in which a solution of hCG powder dissolved in a pharmaceutically suitable buffer is orally administered.

3. The method of claim 1 in which a solution of hCG powder dissolved in physiological saline is orally administered.

4. The method of claim 1 in which a solution of 500 IU of hCG powder per millimeter of physiological saline is orally administered.
